SEQUENCE LISTING (5 sequences)

SEQ ID NO: 1 - DNA, 3543 bp, synthetic construct
  misc_feature 1..3543: note = Human cDNA
  source 1..3543: mol_type = other DNA
agacactgctacatgttcttcataaattaacaccctcataaaggtaaaccaagaaggttatcctcaatcatctggtatcaatatataattatttttcacatttctgttactttttaatgagatttgaggttgttctgtgattgttatcagaattaccaatgcacaaaagccagaatgtatttggaaactagaagagctatttttgttttttggatttttctccaagttcaaggaaccaaagatatctccattaacatataccattctgaaactaaagacatagacaatcccccaagaaatgaaacaactgaaagtactgaaaaaatgtacaaaatgtcaactatgagacgaatattcgatttggcaaagcatcgaacaaaaagatccgcatttttcccaacgggggttaaagtctgtccacaggaatccatgaaacagattttagacagtcttcaagcttattatagattgagagtgtgtcaggaagcagtatgggaagcatatcggatctttctggatcgcatccctgacacaggggaatatcaggactgggtcagcatctgccagcaggagaccttctgcctctttgacattggaaaaaacttcagcaattcccaggagcacctggatcttctccagcagagaataaaacagagaagtttccctgacagaaaagatgaaatatctgcagagaagacattgggagagcctggtgaaaccattgtcatttcaacagatgttgccaacgtctcacttgggcctttccctctcactcctgatgacaccctcctcaatgaaattctcgataatacactcaacgacaccaagatgcctacaacagaaagagaaacagaattcgctgtgttggaggagcagagggtggagctcagcgtctctctggtaaaccagaagttcaaggcagagctcgctgactcccagtccccatattaccaggagctagcaggaaagtcccaacttcagatgcaaaagatatttaagaaacttccaggattcaaaaaaatccatgtgttaggatttagaccaaagaaagaaaaagatggctcaagctccacagagatgcaacttacggccatctttaagagacacagtgcagaagcaaaaagccctgcaagtgacctcctgtcttttgattccaacaaaattgaaagtgaggaagtctatcatggaaccatggaggaggacaagcaaccagaaatctatctcacagctacagacctcaaaaggctgatcagcaaagcactagaggaagaacaatctttggatgtggggacaattcagttcactgatgaaattgctggatcactgccagcctttggtcctgacacccaatcagagctgcccacatcttttgctgttataacagaggatgctactttgagtccagaacttcctcctgttgaaccccagcttgagacagtggacggagcagagcatggtctacctgacacttcttggtctccacctgctatggcctctacctccctgtcagaagctccacctttctttatggcatcaagcatcttctctctgactgatcaaggcaccacagatacaatggccactgaccagacaatgctagtaccagggctcaccatccccaccagtgattattctgcaatcagccaactggctctgggaatttcacatccacctgcatcttcagatgacagccgatcaagtgcaggtggcgaagatatggtcagacacctagatgaaatggatctgtctgacactcctgccccatctgaggtaccagagctcagcgaatatgtttctgtcccagatcatttcttggaggataccactcctgtctcagctttacagtatatcaccactagttctatgaccattgcccccaagggccgagagctggtagtgttcttcagtctgcgtgttgctaacatggccttctccaacgacctgttcaacaagagctctctggagtaccgagctctggagcaacaattcacacagctgctggttccatatctacgatccaatcttacaggatttaagcaacttgaaatacttaacttcagaaacgggagtgtgattgtgaatagcaaaatgaagtttgctaagtcagtgccgtataacctcaccaaggctgtgcacggggtcttggaggattttcgttctgctgcagcccaacaactccatctggaaatagacagctactctctcaacattgaaccagctgatcaagcagatccctgcaagttcctggcctgcggcgaatttgcccaatgtgtaaagaacgaacggactgaggaagcggagtgtcgctgcaaaccaggatatgacagccaggggagcctggacggtctggaaccaggcctctgtggccctggcacaaaggaatgcgaggtcctccagggaaagggagctccatgcaggttgccagatcactctgaaaatcaagcatacaaaactagtgttaaaaagttccaaaatcaacaaaataacaaggtaatcagtaaaagaaattctgaattactgaccgtagaatatgaagaatttaaccatcaagattgggaaggaaattaaaaactgaaaatgtacaattatcatttaggctatctcaagagagatgatttgccttctcaaggaaaatggagacaggcatattcatgggtcatcaaaatccagacatacagtcaacactgagaatcagcacacaccatatttcaaatatagaagagtcatgtacttggcaaccagtaaattctgaagaaaaagacacttacttattattaaaaccccaaatgcaatcagcgaaacatatttttactattcttggatgatagtcaaaatgatcataagccaggtttgcttccaccttccctgaaaattttactcacagatcatttgcaacaagcatagcttacttattgtttagggactgaacaatttattgggaagcaaactctttatatgctagaaagtacatttaaaagatgactacttacgcagggagatgcaggtctctctaaacgcatgaatgtatgtagtgtgtaggcactgtagtgagtgtatatatgctccacactacgtctgataaacacaaacctcagtattcagttattaggcacactagttttatacgcaactactgcttacatagtagactgttttgttgccaataatctttgaattgttctttaaaagaaactgaggttcagatacacataccatggaaaaatcttacttttcttgttactacacaaagctattttaaagaagatgctatgttgggagaagggcgaagttgtactatatgacataatcaattcctgtcttccaccacagatgaacaatgtcttcctataataacttcagaatatttcctcacaccaactttagtgtgtgtatacctagactggcatcaatgtaatccaattcagtccattttttatgtgctgcttaatgaaaatgaatctgtctctatctctttcaaatgttctaatatcagatatatttgctacaaaccttaatttcaataaagtaaaagtgatatactca

SEQ ID NO: 2 - DNA, 3726 bp, synthetic construct
  misc_feature 1..3726: note = Human cDNA
  source 1..3726: mol_type = other DNA
atgattatgtttcctctttttgggaagatttctctgggtattttgatatttgtcctgatagaaggagactttccatcattaacagcacaaacctacttatctatagaggagatccaagaacccaagagtgcagtttcttttctcctgcctgaagaatcaacagacctttctctagctaccaaaaagaaacagcctctggaccgcagagaaactgaaagacagtggttaatcagaaggcggagatctattctgtttcctaatggagtgaaaatctgcccagatgaaagtgttgcagaggctgtggcaaatcatgtgaagtattttaaagtccgagtgtgtcaggaagctgtctgggaagccttcaggactttttgggatcgacttcctgggcgtgaggaatatcattactggatgaatttgtgtgaggatggagtcacaagtatatttgaaatgggcacaaattttagtgaatctgtggaacatagaagcttaatcatgaagaaactgacttatgcaaaggaaactgtaagcagctctgaactgtcttctccagttcctgttggtgatacttcaacattgggagacactactctcagtgttccacatccagaggtggacgcctatgaaggtgcctcagagagcagcttggaaaggccagaggagagtattagcaatgaaattgagaatgtgatagaagaagccacaaaaccagcaggtgaacagattgcagaattcagtatccaccttttggggaagcagtacagggaagaactacaggattcctccagctttcaccaccagcaccttgaagaagaatttatttcagaggttgaaaatgcatttactgggttaccaggctacaaggaaattcgtgtacttgaatttaggtcccccaaggaaaatgacagtggcgtagatgtttactatgcagttaccttcaatggtgaggccatcagcaataccacctgggacctcattagccttcactccaacaaggtggaaaaccatggccttgtggaactggatgataaacccactgttgtttatacaatcagtaacttcagagattatattgctgagacattgcagcagaattttttgctggggaactcttccttgaatccagatcctgattccctgcagcttatcaatgtgagaggagttttgcgtcaccaaactgaagatctagtttggaacacccaaagttcaagtcttcaggcaacgccgtcatctattctggataatacctttcaagctgcatggccctcagcagatgaatccatcaccagcagtattccaccacttgatttcagctctggtcctccctcagccactggcagggaactctggtcagaaagtcctttgggtgatttagtgtctacacacaaattagcctttccctcgaagatgggcctcagctcttccccagaggttttagaggttagcagcttgactcttcattctgtcaccccggcagtgcttcagactggcttgcctgtggcttctgaggaaaggacttctggatctcacttggtagaagatggattagccaatgttgaagagtcagaagattttctttctattgattcattgccttcaagttcattcactcaacctgtgccaaaagaaacaataccatccatggaagactctgatgtgtccttaacatcttcaccatatctgacctcttctataccttttggcttggactccttgacctccaaagtcaaagaccaattaaaagtgagccctttcctgccagatgcatccatggaaaaagagttaatatttgacggtggtttaggttcagggtctgggcaaaaggtagatctgattacttggccatggagtgagacttcatcagagaagagcgctgaaccactgtccaagccgtggcttgaagatgatgattcacttttgccagctgagattgaagacaagaaactagttttagttgacaaaatggattccacagaccaaattagtaagcactcaaaatatgaacatgatgacagatccacacactttccagaggaagagcctcttagtgggcctgctgtgcccatcttcgcagatactgcagctgaatctgcgtctctaaccctccccaagcacatatcagaagtacctggtgttgatgattactcagttaccaaagcacctcttatactgacatctgtagcaatctctgcctctactgataaatcagatcaggcagatgccatcctaagggaggatatggaacaaattactgagtcatccaactatgaatggtttgacagtgaggtttcaatggtaaagccagatatgcaaactttgtggactatattgccagaatcagagagagtttggacaagaacttcttccctagagaaattgtccagagacatattggcaagtacaccacagagtgctgacaggctctggttatctgtgacacagtctaccaaattgcctccaaccacaatctccaccctgctagaggatgaagtaattatgggtgtacaggatatttcgttagaactggaccggataggcacagattactatcagcctgagcaagtccaagagcaaaatggcaaggttggtagttatgtggaaatgtcaacaagtgttcactccacagagatggttagtgtggcttggcccacagaaggaggagatgacttgagttatacccagacttcaggagctttggtggttttcttcagcctccgagtgactaacatgatgttttcagaagatctgtttaataaaaactccttggagtataaagccctggagcaaagattcttagaattgctggttccctatctccagtcaaatctcacggggttccagaacttagaaatcctcaacttcagaaatggcagcattgtggtgaacagtcgaatgaagtttgccaattctgtccctcctaacgtcaacaatgcggtgtacatgattctggaagacttttgtaccactgcctacaataccatgaacttggctattgataaatactctcttgatgtggaatcaggtgatgaagccaacccttgcaagtttcaggcctgtaatgaattttcagagtgtctggtcaacccctggagtggagaagcaaagtgcagatgcttccctggatacctgagtgtggaagaacggccctgtcagagtctctgtgacctacagcctgacttctgcttgaatgatggaaagtgtgacattatgcctgggcacggggccatttgtaggtgccgggtgggtgagaactggtggtaccgaggcaagcactgtgaggaatttgtgtctgagcccgtgatcataggcatcactattgcctccgtggttggacttcttgtcatcttttctgctatcatctacttcttcatcaggactctccaagcacaccatgacaggagtgaaagagagagtcccttcagtggctccagcaggcagcctgacagcctctcatctattgagaatgctgtgaagtacaaccccgtgtatgaaagtcacagggctggatgtgagaagtatgagggaccctatcctcagcatcccttctacagctctgctagcggagacgtgattggtgggctgagcagagaagaaatcagacagatgtatgagagcagtgagctttccagagaggaaattcaagagagaatgagagttttggaactgtatgccaatgatcctgagtttgcagcttttgtgagagagcaacaagtggaagaggtttaa

SEQ ID NO: 3 - DNA, 20 bp, synthetic construct
  misc_feature 1..20: note = Annealing sequence
  source 1..20: mol_type = other DNA
ccctgcactgcttccccaaa

SEQ ID NO: 4 - DNA, 35 bp, Homo sapiens
  source 1..35: mol_type = genomic DNA
ggacatcaagaagggacgtgacgaaggggttttcc

SEQ ID NO: 5 - DNA, 35 bp, Homo sapiens
  source 1..35: mol_type = genomic DNA
cctgtagttcttccctgcactgcttccccaaaagg

(19) United States
(12) Patent Application Publication
(10) Pub. No.: US 2023/0151327 A1
(43) Pub. Date: May 18, 2023

(54) RETINAL ORGANOID MODEL SYSTEMS

(71) Applicant: Wisconsin Alumni Research Foundation, Madison, WI (US)

(72) Inventors: David Gamm, Waunakee, WI (US); Steven Mayerl, Colorado Spring, CO (US)

(21) Appl. No.: 17/820,488

(22) Filed: Aug. 17, 2022

Related U.S. Application Data
(60) Provisional application No. 63/238,415, filed on Aug. 30, 2021.

(51) Int. Cl. C12N 5/079

(52) U.S. Cl. CPC C12N 5/0621; C12N 2503/02; C12N 2503/04; C12N 2513/00

(57) ABSTRACT

This disclosure relates to genetically engineered retinal organoids that can be used to test therapeutic treatments.

### CROSS REFERENCE TO RELATED APPLICATIONS

This application is a United States utility patent application, which claims the benefit of U.S. provisional application No. 63/238,415, filed Aug. 30, 2021, which is hereby incorporated by reference in its entirety.

### STATEMENT REGARDING GOVERNMENT SUPPORT

This invention was made with government support under EY021218 and EY031230 awarded by the National Institutes of Health. The government has certain rights in the invention.

### REFERENCE TO AN ELECTRONIC SEQUENCE LISTING

A computer readable form of the Sequence Listing is filed with this application by electronic submission and is incorporated into this application by reference in its entirety. The Sequence Listing is contained in the file created on Aug. 16, 2022, having the file name “21-0000-US.xml” and is 14 kilobytes in size.

### BACKGROUND OF THE DISCLOSURE

### Field of the Invention

This disclosure relates to genetically engineered retinal organoids and uses thereof.

### Description of Related Art

A multitude of human disorders is directly attributable to genetic mutations; the retina has been at the forefront of gene therapy testing for a host of reasons, and companies that are not ultimately interested in eye conditions nonetheless often “start” in the eye due to its clear advantages. For example, the retina is easily accessible using standard surgical procedures and represents relatively low risk for experimentation. Many standardized, noninvasive imaging and functional tests are available for monitoring treatment effects in retinal models, and many different genes and gene mutations (e.g. missense, nonsense, frameshift, cryptic splice variants, coding/non-coding) lead to dysfunction and blindness, with significant genotype-phenotype heterogeneity. Nevertheless, there remains a need for robust and clinically relevant in vitro models that can be used for testing of gene therapy in a laboratory setting.

The capacity to generate bona fide fetal-stage neural retinal cell types and tissues from human embryonic and induced pluripotent stem cells (hESCs and hiPSCs, respectively; collectively, human pluripotent stem cells (hPSCs)) has spurred their use for in vitro disease modeling. Most of these studies employ hPSC differentiation methods that propagate retinal progeny as isolated 3D optic vesicle-like structures (OVs), also known as retinal organoids, in suspension culture. Benefits of these 3D culture techniques include attainment of high percentages of retinal cell types with low or absent non-retinal contamination and a predilection to self-organize into highly mature tissue structures. However, further improvements in model systems are needed to provide easier and faster testing of novel therapeutics for genetic disorders, and there remains an urgent need to develop model systems that mimic genetic disorders, to facilitate identification of new, clinically relevant treatments for genetic disorders.

### BRIEF SUMMARY

As described herein, in a first aspect, the present disclosure provides a retinal organoid model system, having a population of human pluripotent stem cell (hPSC)—derived photoreceptor (PR) cells. The PR cells are adapted to elaborate an interphotoreceptor matrix (IPM) with visible outer segments on a surface thereof upon restoration of function of a gene encoding a structural component of the IPM.

In one embodiment of the first aspect, the hPSC is a human embryonic stem cell (hESC) or a human induced pluripotent stem cell (hiPSC). In one embodiment of the first aspect, the function of the gene is restorable by administration of a therapeutic treatment to the retinal organoid. In one embodiment, the therapeutic treatment comprises a protein, a virus, a RNA molecule, a DNA molecule, or a small molecule. In one embodiment, the therapeutic treatment further comprises a gene editor, a base editor, an RNA editor, a small molecule targeting DNA/RNA, or a cell therapy. In another embodiment of the first aspect, the hPSC is a hiPSC. In one embodiment, the hiPSC is derived from a patient with a naturally occurring mutation in a gene encoding a structural component of the IPM. In one embodiment, the naturally occurring mutation is one or more of a missense mutation, a nonsense mutation, a frameshift mutation, a cryptic slice variant mutation, a coding mutation, or non-coding mutation. In one embodiment, the hiPSC is a recombinant hiPSC comprising a genetically engineered mutation in at least one allele in the gene encoding a structural component of the IPM. In one embodiment, the genetically engineered mutation is one or more of a missense mutation, a nonsense mutation, a frameshift mutation, a cryptic slice variant mutation, a coding mutation, or non-coding mutation. In another embodiment of the first aspect, the hPSC is a hESC. In one embodiment, the hESC is H9, H1, H7, BGO1, BG02, HES-3, HES-2, HSF-6, HUES9, HUES7, or 16. In one embodiment, the hESC is a recombinant hESC comprising a genetically engineered mutation in at least one allele in the gene encoding a structural component of the IPM. In one embodiment, the genetically engineered mutation is one or more of a missense mutation, a nonsense mutation, a frameshift mutation, a cryptic slice variant mutation, a coding mutation, or non-coding mutation. In another embodiment of the first aspect, the gene encoding a structural component of the IPM is IMPG1 or IMPG2. In one embodiment, the gene encoding a structural component of the IPM is IMPG2.

In a second aspect, the present disclosure provides a retinal organoid model system, including a population of human pluripotent stem cell (hPSC)—derived photoreceptor (PR) cells. The PR cells include a recombinant gene encoding a structural component of an interphotoreceptor matrix (IPM). The recombinant gene includes at least one of a first non-functional allele having a first engineered genetic mutation, and/or a second non-functional allele having a second engineered genetic mutation. The restoration of function of at least one of the first and second alleles produces an IPM containing visible outer segments on a surface of the PR cells.

In one embodiment of the second aspect, the hPSC is a human embryonic stem cell (hESC) or a human induced pluripotent stem cell (hiPSC). In one embodiment of the second aspect, the function of the gene is restored by administration of a therapeutic treatment to the retinal organoid. In one embodiment, the therapeutic treatment comprises a protein, a virus, a RNA molecule, a DNA molecule, or a small molecule. In one embodiment, the therapeutic treatment comprises a gene editor, a base editor, an RNA editor, a small molecule targeting DNA/RNA, or a cell therapy. In one embodiment of the second aspect, the hPSC is a hiPSC. In one embodiment of the second aspect, the first engineered genetic mutation is one or more of a missense mutation, a nonsense mutation, a frameshift mutation, a cryptic slice variant mutation, a coding mutation, or non-coding mutation. In one embodiment of the second aspect, the second engineered genetic mutation is one or more of a missense mutation, a nonsense mutation, a frameshift mutation, a cryptic slice variant mutation, a coding mutation, or non-coding mutation. In one embodiment of the second aspect, the first engineered genetic mutation and the second engineered genetic mutation are the same mutation. In another embodiment of the second aspect, the hPSC is a hESC. In one embodiment, the hESC is H9, H1, H7, BGO1, BG02, HES-3, HES-2, HSF-6, HUES9, HUES7, or 16. In another embodiment of the second aspect, the gene encoding a structural component of the IPM is IMPG1 or IMPG2. In one embodiment, the gene encoding a structural component of the IPM is IMPG2.

In a third aspect, the present disclosure provides a method of testing for the efficacy of a therapeutic treatment using the retinal organoid model system of any one of the preceding aspects or embodiments thereof. The method includes administering to the retinal organoid model system a candidate therapeutic treatment for restoring function of the gene encoding a structural component of the IPM; visualizing the photoreceptor outer segments within the IPM of the retinal organoid model system; and detecting a change in the IPM. A change in production and/or maintenance of microscopically visible photoreceptor outer segments within the IPM of the retinal organoid model system indicates that the therapeutic treatment was effective in the restoration of function of the gene encoding a structural component of the IPM, and no change in the production and/or maintenance of microscopically visible photoreceptor visible outer segments within the IPM of the retinal organoid model system indicates that the therapeutic treatment was not effective.

In one embodiment of the third aspect, the method further comprises visualizing the IPM prior to the administration of the candidate therapeutic. In one embodiment of the third aspect, the visualizing the IPM of the retinal organoid model system comprises qualitatively observing or quantifying the presence of visible photoreceptor outer segments on the surface of the PR cells.

In a fourth aspect, the present disclosure provides a method of testing for an effective therapeutic treatment for a genetic mutation, including producing a retinal organoid model system comprising a population of human pluripotent stem cell (hPSC)—derived photoreceptor (PR) cells adapted to express an interphotoreceptor matrix (IPM) with visible outer segments on a surface thereof upon restoration of function of a gene encoding a structural component of the IPM. The gene encoding the structural component of the IPM comprises a predetermined or pre-existing genetic mutation. The method further includes administering a candidate therapeutic treatment to the retinal organoid model system and assessing the retinal organoid model system for presence or absence of visible outer segments within the IPM. The presence of visible outer segments of the IPM indicates that the candidate therapeutic treatment restores the function of the gene encoding the structural component of the IPM by effectively treating the predetermined genetic mutation.

In one embodiment of the fourth aspect, the assessing further comprises using the qualitative observation or quantification of the visible outer segments on the surface of the PR cells to determine the presence of absence of a change in the IPM, and wherein an increase in the presence or quantity of the visible outer segments indicates the presence of a change in IPM of the retinal organoid model system. In one embodiment of the fourth aspect, the therapeutic treatment includes a protein, a virus, a RNA molecule, a DNA molecule, a gene therapy, or a small molecule. In one embodiment, the therapeutic treatment comprises a gene editor, a base editor, an RNA editor, a small molecule targeting DNA/RNA, or a cell therapy. In one embodiment, the virus comprises an adeno associated viral vector (AAV) or a lentivirus. In one embodiment, the therapeutic treatment comprises a genome or base editing technology, a nanoparticle, or a cellular delivery mechanism. In one embodiment of the fourth aspect, the predetermined genetic mutation comprises a missense mutation, a nonsense mutation, a frameshift mutation, a cryptic slice variant mutation, a coding mutation, or non-coding mutation, or a knockout mutation. In one embodiment of the fourth aspect, the candidate therapeutic treatment is a candidate for the treatment of a genetic disease. In one embodiment, the genetic disease is cystic fibrosis, sickle-cell anemia, hemochromatosis, Huntington's disease, Duchenne's muscular dystrophy Tay-Sachs disease, Angelman syndrome, Ankylosing spondylitis, Marfan syndrome, or Thalassemia.

In a fifth aspect, the present disclosure provides a method of making a retinal organoid model system, including: engineering one or more genetic mutations in a gene encoding a structural component of the interphotoreceptor matrix (IPM) in a population of human pluripotent stem cells (hPSCs); and inducing the hPSCs to differentiate along a retinal lineage, wherein the differentiation results in a three-dimensional (3D) retinal organoid comprising photoreceptor (PR) cells adapted to express visible outer segments within the IPM on a surface thereof upon restoration of function of the gene encoding a structural component of the IPM.

In a sixth aspect, the present disclosure provides a method of making a retinal organoid model system, including: obtaining a tissue sample from a subject having a mutation in a gene encoding a structural component of the interphotoreceptor matrix (IPM); establishing a population of human induced pluripotent stem cells (hiPSCs) from the tissue sample; and inducing the hiPSCs to differentiate along a retinal lineage, wherein the differentiation results in a three-dimensional (3D) retinal organoid comprising photoreceptor (PR) cells. The PR cells are adapted to express an interphotoreceptor matrix (IPM) structural protein, and the restoration of the IPM structure allows the production and maintenance of visible photoreceptor outer segments on the surface of the retinal organoids.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more non-limiting embodiment(s) of the disclosure, and together with the description serve to explain the principles and operation of the disclosure.

FIGS. 1A-1D show light microscopic categorization of differentiating hPSC-ROs: (FIG. 1A) Stage 1 hPSC-ROs; (FIG. 1B) Stage 2 hPSC-ROs; (FIGS. 1C and 1D) Stage 3 hPSC-RO. FIG. 1D is a magnified image of the boxed area in FIG. 1C showing photoreceptor outer segments (bracketed). Scale bars are 100 μm (FIGS. 1A and 1C) and 25 μm (FIGS. 1B and 1D).

FIGS. 2A-2E show differentiation of hPSC-ROs containing IMPG2 mutations result in a lack of photoreceptor outer segments: (FIG. 2A) iPSC IMPG2^{Y254C/A805(fs)Ter} (FIG. 2B) iPSC IMPG2^{Y254C/+}, (FIG. 2C) iPSC IMPG2^{-/-}, (FIG. 2D) H9 IMPG2^{Y254C/Y254C}, and (FIG. 2E) H9 IMPG2^{-/-}. Scale bars=25 μm.

FIGS. 3A-3D show stage 3 gene-corrected IMPG2 hPSC-ROs display photoreceptor outer segments: (FIGS. 3A, 3B) iPSC IMPG2^{+/+} ROs demonstrate full restoration of photoreceptor outer segments along their surface. A magnified image of the RO shown in (FIG. 3A) is shown in (FIG. 3B). (FIGS. 3C, 3D) show wildtype H9 ROs. Scale Bars=100 μm (FIGS. 3A, 3C); 25 μm (FIGS. 3B, 3D).

FIGS. 4A-4E show light microscopic imaging of ROs derived from mixed cultures of wild type (WT) and mutant (MT) IMPG2 hPSCs. FIG. 4A shows a 100% WT control hPSC-RO, FIG. 4B shows a 50:50 WT:MT hPSC-RO; FIG. 4C shows a 20:80 WT:MT hPSC-RO, FIG. 4D shows a 5:95 WT:MT hPSC-RO, and FIG. 4E shows a 100% MT IMPG2 hPSC-RO. Scale Bar=20 μm.

FIG. 5 shows fluorescent confocal imaging of a mixed WT (CRX-TdTomato):MT IMPG2 hPSC-RO. The WT portion (left of the dashed line) of the IMPG2 hPSC-RO shows robust expression of IMPG2, which largely corresponds with CRX-TdTomato expression. The MT portion (right of the dashed line) shows little to no staining for either IMPG2 or CRX-TdTomato.

FIG. 6 shows one example of a base editing approach to correct for mutations in IMPG2. The sequences depicted from top to bottom in the figure correspond to SEQ ID NOs: 3-5, respectively.

### DETAILED DESCRIPTION

### Definitions

Unless defined otherwise, all technical and scientific terms used herein have the meaning commonly understood by a person skilled in the art to which this disclosure belongs. Within this application, unless otherwise stated, the techniques utilized may be found in any of several conventional references such as: Molecular Cloning: A Laboratory Manual (Sambrook et al., 1989, Cold Spring Harbor Laboratory Press); Gene Expression Technology (Methods in Enzymology, Vol. 185, edited by D. Goeddel, 1991, Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis et al. 1990. Academic Press, San Diego, Calif.); Culture of Animal Cells: A Manual of Basic Technique, 2^{nd} Ed. (R.I. Freshney. 1987. Liss, Inc. New York, N.Y.); Gene Transfer and Expression Protocols, pp. 109-128, ed. E. J. Murray, The Humana Press Inc., Clifton, N.J.); and the Ambion 1998 Catalog (Ambion, Austin, Tex.).

As used herein, ranges and amounts can be expressed as “about” a particular value or range. About also includes the exact amount. For example, “about 5%” means “about 5%” and also “5%.” The term “about” can also refer to +10% of a given value or range of values. Therefore, about 5% also means 4.5% -5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

In this disclosure, the terms “comprises,” “comprising,” “containing,” “having,” and the like can have the meaning ascribed to them in U.S. Patent law and can mean “includes,” “including,” and the like. As used herein, the terms “determining,” “assessing,” “assaying,” “measuring,” “detecting,” and “identifying” refer to both quantitative and qualitative determinations, and as such, the terms can be used interchangeably, but where appropriate their quantitative or qualitative nature will be understood in context by the skilled artisan.

As used herein, the singular forms “a,” “an,” and “the” include plural referents unless the context clearly dictates otherwise.

As used herein, a “retinal organoid” is an in vitro generated cell cluster that mimics cellular ultrastructure and function of retinal tissue.

As used herein, a “retinal organoid model system” refers to a retinal organoid which can be used to identify or evaluate therapeutic treatments for genetic mutations.

As used herein, “pluripotency” refers to a cell's ability to differentiate into cells of all three germ layers.

As used herein, the term human “pluripotent stem cell” (hPSC) refers to a cell capable of continued self-renewal and capable, under appropriate conditions, of differentiating into cells of all three germ layers. Examples of hPSCs include human embryonic stem cells (hESCs) and human induced pluripotent stem cells (hiPSCs). As used herein, “iPS cells” refer to cells that are substantially genetically identical to their respective differentiated somatic cell of origin and display characteristics similar to higher potency cells, such as embryonic stem (ES) cells, as described herein. The cells can be obtained by reprogramming non-pluripotent (e.g., multipotent or somatic) cells.

“Photoreceptor cells” or “PR cells,” as used herein, are a specialized type of neuroepithelial cell that is capable of visual phototransduction and comprises an inner-segment and an outer-segment. There are two types of PR cells: rods and cones. Rods are adapted for low-light vision, to view in grayscale, and cones are adapted for daylight vision, to view in color.

As used herein, the term “elaborate” means to develop, express, or display. For example, PR cells are adapted to elaborate an interphotoreceptor matrix (IPM), which indicates that they are adapted to develop, express, and/or display an IPM.

The term “interphotoreceptor matrix” or “IPM,” as used herein, is a highly organized structure, which is located between the photoreceptors and the retinal pigment epithelium (RPE). The IPM is an essential component of healthy, normal retina and is critical for trafficking metabolites, retinal attachment, and photoreceptor alignment.

As used herein, an “outer segment” is the part of the PR cell closest to the brain, and farthest from the field of view. The outer segment is the part of the PR cell that absorbs light.

As used herein, the term “wild-type” refers to a normal, healthy, and/or un-changed state. For example, a wild-type phenotype results when an unmutated gene is expressed. Further, a wild-type gene refers to a gene that when expressed results in a normal, functional phenotype.

As used herein, the term “phenotype” refers to a set of observable characteristics resulting from a genotype. For example, a wild-type phenotype refers to a phenotype that is the result of normal expression of a wild-type gene or set of genes.

As used herein, an “allele” is any one of two or more versions of a gene that can occur alternatively at a given site (locus) on a chromosome. Alleles can occur in pairs, or there may be multiple alleles affecting the expression (phenotype) of a particular trait attributable to the gene responsible for the trait.

As used herein, the terms “visible outer segment” or “visible IPM phenotype” refer to an IPM having hair-like photoreceptor structures on the outer segments of the PR cells that can be visually observed, for example, with the use of a microscope.

The term “restoration of function,” when used in the context of a partially functional or non-functional gene, refers to a restoration of a wild-type phenotype when the gene is expressed. For example, a restoration of function of a gene can occur when a mutation in the gene is functionally repaired (not necessarily returning the gene itself to a wild-type, non-mutated form) by a therapeutic agent to restore a wild-type phenotype. It is contemplated herein that a restoration of function can be partial, that is, not a complete return to a fully wild-type function (or phenotype). In this context, a restoration of function can be observed as an improvement in function that can be clinically relevant. For example, a partial restoration of function of a gene can lead to a partial restoration of vision and/or a decrease in a rate of loss of vision in an individual treated with a therapeutic agent, as contemplated herein.

The term “mutation” is defined as a change to a sequence or structure of a gene, resulting in a variant form of the gene or a part thereof that may be transmitted to subsequent generations. Mutations can be caused, for example, by the alteration of single base units in DNA, or the deletion, insertion, or rearrangement of larger sections of the gene(s).

A “naturally occurring” genetic mutation is a mutation in a gene, or a part thereof, which occurs spontaneously in a population. For example, naturally occurring mutations can be heritable and can be associated with genetic diseases, such as retinitis pigmentosa.

As used herein, the term “recombinant,” as used herein, refers to a gene, cell, or tissue that been purposefully genetically modified or genetically engineered. The term “genetically engineered” when used in the context of a gene, refers to a gene or a part thereof that is not naturally occurring and due to genetic manipulation of one or both alleles of the gene. For example, a gene with a non-functional allele can be the result of one or more engineered genetic mutations that have been introduced into the allele. Genetically engineered mutations can be predetermined or can be random and can lead to changes in a gene's expression levels or expressed phenotype. Cells and/or tissues that include one or more genetically engineered genes also considered to be recombinant or genetically engineered.

As used herein, a “predetermined genetic mutation” is a genetic mutation for which the genetic sequence is known. For example, a predetermined genetic mutation can be added to a gene to disrupt the function of the gene. In a specific example, a predetermined genetic mutation can be introduced into a gene that encodes a structural element of the IPM in a retinal organoid. The mutation can disrupt the expression of the gene and thereby compromise the IPM structure leading to a loss of the visible IPM phenotype. Examples of predetermined genetic mutations include one or more of a missense mutation, a nonsense mutation, a frameshift mutation, a cryptic slice variant mutation, a coding mutation, or non-coding mutation, and/or a knockout mutation.

As used herein, an “effective or sufficient amount” or “therapeutically effective amount” is an amount of an agent, such as a therapeutic agent, sufficient to evoke a specified cellular effect according to the present disclosure. For example, an effective or sufficient amount of a therapeutic agent effective for treating a genetic mutation is the amount of the therapeutic agent which results in a partial or full restoration of function of the gene when administered to a subject.

As used herein, a “therapeutic treatment” refers to treatment with a therapeutic agent to provide a positive clinical effect or therapeutic benefit, such as the restoration of function of a gene or a wild-type phenotype.

A “candidate therapeutic treatment” is an experimental treatment for which the effectiveness of the treatment has not been firmly established.

As used herein, “therapeutic agent” can refer to a substance, such as a chemical, compound, and/or pharmaceutical composition that when administered to a subject in need thereof in a therapeutically effective amount provides a therapeutic benefit to the subject having a particular disease or disorder being treated. Other examples of therapeutic agents can include genetically modified cells for use in cell therapies.

As used herein, “therapeutic benefit” refers to the eradication or amelioration of the underlying disease being treated and/or eradication or amelioration of one or more of the symptoms associated with the underlying disease such that a subject being treated with the therapeutic agent reports an improvement in feeling or condition, notwithstanding that the subject may still be afflicted with the underlying disease. Further examples of therapeutic benefit include partial or full restoration of function of a gene and/or partial or full restoration of a phenotype. Still further examples of therapeutic benefit include partial or full cessation of a loss of function associated with a particular disease. In a specific example, a person who experiences a therapeutic benefit may experience improved or restored vision or a decrease in a rate of vision loss associated with retinitis pigmentosa.

By “subject” or “patient” is meant a mammal, including, but not limited to, a human, such as a human patient, a non-human primate, or a non-human mammal, such as a bovine, equine, canine, ovine, or feline animal.

As used herein, the terms “treat,” “treating,” “treatment,” and the like refer to reducing, diminishing, lessening, alleviating, abrogating, or ameliorating a disorder and/or one or more symptoms associated therewith. It will be appreciated that, although not precluded, treating a disorder or condition does not require that the disorder, condition, or symptoms associated therewith be completely eliminated.

Overview

The present disclosure provides retinal organoid model systems for identifying and/or assessing the efficacy of therapeutic treatments for genetic mutations. The retinal organoid model systems can include a genetic mutation that disrupts the IPM resulting in the loss of the visible hair-like, outer segment structures of the photoreceptors. This loss of outer segments of PR cells is a readily visible phenotype indicating the presence of the genetic mutation. A partial or full restoration of the visible outer segments of the IPM by administration of a candidate therapeutic treatment indicates that the candidate therapeutic treatment is effective for treating the genetic mutation. Therefore, the retinal organoid model systems of the present disclosure provide a means for rapidly assessing the efficacy of candidate therapeutic treatments for genetic diseases. Moreover, while the model systems are retinal organoid-based, the genetic mutations that can be tested are not limited to those that adversely affect the eye. Any genetic mutation can be introduced into the systems to cause the loss of the visible outer segments of the IPM, as described elsewhere herein. Therefore, candidate therapeutic treatments being considered for treatment of other genetic diseases outside of the eye caused be specific mutations can be tested in the systems provided herein. As a result, the present disclosure introduces a powerful new tool for the rapid, qualitative assessment of new, clinically relevant treatments for genetic disorders.

### Retinal Organoids

In one embodiment, retinal organoids of the present disclosure are cell clusters that mimic the cellular ultrastructure and function of retinal tissue. The cell clusters form isolated, three-dimensional (3D) optic vesicle-like structures (OVs) which have fully laminated 3D retinal tissues including 3D retinal cups and retinal pigment epithelium (RPE) that include retinal ganglion cells, horizontal cells, amacrine cells, bipolar cells, Mueller cells, and photoreceptor (PR) cells (i.e., rods and cones). In a healthy or wild-type retinal organoid, PR cells with wild-type IPM result in visible outer segments on the IPM surface. The visible outer segments, which look like hair-like structures, can be easily visualized using any type of suitable microscopy and can serve as a visible phenotype which indicates a healthy, wild-type retinal organoid.

Methods for visualizing the outer segments and the IPM can include, but are not limited to, optical microscopy, electron microscopy, and scanning probe microscopy. In various example of optical microscopy, include bright field, confocal and fluorescence.

The retinal organoids of the present disclosure can be produced using human pluripotent stem cells (hPSCs). For example, hPSCs can be induced to develop into the different cell types present in the retinal organoid model system through a differential protocol as outlined in Meyer et al. (2009, PNAS, 106(39): 16698-16703), which is incorporated herein by reference. In various embodiments, the hPSCs can be human embryonic stem cells (hESCs). In some embodiments, the hPSCs can be human induced pluripotent stem cells (hiPSCs) (Zhong et al., 2014, Nature Communications, 5: 4047). In further embodiments, contemplated retinal organoid model systems can be produced using a combination of hESCs and hiPSCs. Non-limiting examples of hESCs that can be used include WA09, WA01, WA07, BG01, BG02, HES-3, HES-2, HSF-6, HUES9, HUES7, and 16 embryonic stem cell lines (Thomson et al., 1998, Science, 282, no. 5391: 1145-1147). A registry of contemplated human embryonic stem cell lines can be found at NIH Human Embryonic Stem Cell Registry.

The retinal organoids of the present disclosure can be produced by methods found in Capowski et al. (2019, Development, 146, no. 1), which is incorporated by reference herein, in its entirety.

The retinal organoids of the model system can be produced, in vitro, to be wild-type retinal organoids or can include naturally occurring genetic mutations or engineered genetic mutations in the genes encoding one or more structural components of the IPM. In wild-type retinal organoids of the model system, the outer segments of the IPM would be microscopically visible and would appear as tiny hair-like structures on the surface of the IPM. In contrast, retinal organoid including one or more genetic mutations in the genes encoding the structural components of the IPM would display a reduction in the number of visible outer segments and/or a total loss of the visible outer segments of the IPM.

The structural components of the IPM comprise RPE, PHAMM, IMPG2, IMPG1, PEDF, CD44, Müller cells, and hyaluronic acid, among other components (Ishikawa, et al., 2015 Experimental Eye Research, Ocular extracellular matrix: Role in development, homeostasis and disease, 13: 3-18).

### Disease Models

Disorders of the retina can be associated with characteristic changes in IPM components. Such changes in IPM can result in changes to the visible phenotype. One example of a disorder of the retina is retinitis pigmentosa (RP). RP is a heterogeneous group of rare inherited retinal degenerative diseases primarily characterized by progressive loss of photoreceptors over years to decades. RP can be caused by mutations in >80 genes involved in the function and maintenance of photoreceptors. In one example of RP, a gene for IMPG2 is mutated and results in loss of outer segments of the PR and eventual death of rods and cones, and subsequently blindness. Compromised expression of the IMPG2 gene in this type of RP results in loss of readily visible outer segments, and can be easily diagnosed based on a lack of visible hair-like outer segments on the IPM surface.

In various embodiments of the disclosure, retinal organoid model systems can include one or more genetic mutations; genetic mutations in one or both of the alleles of a gene; and/or genetic mutations in one or more of the genes which encodes a structural component of the IPM. Such genetic mutation(s) result in a reduction or loss of visible outer segments on the IPM surface. Thus, the presence of one or more mutations, in one or both alleles, in one or more of the genes, which encode a structural component of the IPM, can be visualized based on the outer segment of the IPM phenotype.

In various embodiments, the genes which encode a structural component of the IPM can include IMPG1, IMPG2, Perlecan, HCAM, HAPLN1, HAPLN4, and/or Versican. In a preferred embodiment, the genetically modified structural gene is IMPG2.

In various embodiments of the disclosure, the mutation in the gene encoding a structural component of the IPM can be naturally occurring or genetically engineered and can include one or more of a missense mutation, a nonsense mutation, a frameshift mutation, a cryptic slice variant mutation, a coding mutation, and/or non-coding mutation.

Various examples of naturally occurring mutations in the gene encoding a structural component of the IPM include missense mutations such as Y254C or frameshift mutations such as A805(fs)Ter, or nonsense mutations. Published mutations can be found in papers such as Bandah-Rozenfeld et al. (2010, American Journal of Human Genetics 87 (2): 199-208) and Brandl et al. (2017, Genes 8 (7): 170).

Other natural variants can be found at www.uniprot.org/uniprot/Q9BZV3.

Various examples of an engineered genetic mutation that can be introduced into the gene encoding a structural component of the IPM include nonsense mutations such as Y254C or frameshift mutations such as A805(fs)Ter.

In various embodiments of the disclosure, the retinal organoids of the present disclosure are produced to include a predetermined genetic mutation(s), such as a genetic mutation found in a genetic disease.

In various embodiments, the retinal organoid model system can comprise an engineered genetic mutation in at least one or both alleles of a gene encoding a structural component of the IPM. In one non-limiting example, the retinal organoid model system includes one or more genetically engineered mutations in one or both of IMPG2 alleles.

In various examples of this embodiment, the engineered genetic mutation is IMPG2^{Y254C/A805(fs)Ter}, IMPG2^{Y254C/+}, IMPG2^{-/-}, and/or IMPG2^{Y254C/Y254C}.

There are many genetic diseases caused by well-characterized and reproducible genetic mutations. Retinal organoids of the present disclosure can be produced that harbor genetic mutations in known genetic diseases, and in this way, such retinal organoids can be used to test therapeutic agents intended for treating these diseases. Examples of genetic diseases with genetic mutations contemplated for testing in the retinal organoid model systems of the present disclosure include, but are not limited to, cystic fibrosis, Marfan syndrome, sickle-cell anemia, hemochromatosis, Huntington's disease, Duchenne's muscular dystrophy, Tay-Sachs disease, Angelman syndrome, Ankylosing spondylitis, and Thalassemia.

In one non-limiting example, a retinal organoid model system of the present disclosure can be genetically engineered to include a deletion in one or both alleles of a gene which encodes structural component of the IPM so that the deletion mimics the most common deletion known to cause cystic fibrosis. The retinal organoid model system could then be used to evaluate methods for the restoration of function for the gene and candidate therapeutic treatments for cystic fibrosis.

### Restoration of Function and Therapeutic Treatments

The retinal organoid model systems of the present disclosure are designed to produce a visible phenotype in response to a restoration of function of the one or more gene mutations in the genes which encode a structural component of the IPM. A restoration of function of the gene mutation(s) results in the restoration of the visible hair-like outer segments on the IPM surface. Thus, the restoration of function of the one or more gene mutation(s) in a gene that encodes a structural component of the IPM, can be visualized by the presence of visible outer segments on the IPM surface. Accordingly, retinal organoid model systems of the present disclosure provide an easily visualized phenotype, directly corresponding to the functional genotype of the gene which encodes a structural component of the IPM. This restoration of function can occur via any mechanism suitable for restoration of gene function.

Restoration of visible outer segments on the IPM can include a partial or full increase in the number, size, or density of the outer segments. Restoration does not require a complete return to wild-type appearance (or phenotype) and can include any visible improvement that is clinically relevant. Phenotypic restoration of the outer segments of the IPM can vary according to the mutation(s) in the gene encoding the structural components of the IPM and the mechanism for restoration of gene function.

In one example, gene function is restorable by administration of a therapeutic treatment to the retinal organoid model system. In various embodiments of this example, the therapeutic treatment can include, but is not limited to, treatment with a protein, a virus, an RNA molecule, a DNA molecule, a small molecule, a gene editor, a base editor, an RNA editor, a small molecule targeting DNA/RNA, or a cell therapy, and any combination thereof.

In several non-limiting examples, the candidate therapeutic treatments include gene augmentation, genome editing, base editing, RNA trans-splicing molecules, antisense oligonucleotides, nonsense read-through drugs, and others.

In various non-limiting examples, viruses that can be used include adenoviruses, adeno-associated viruses (AAV), alphaviruses, flaviviruses, herpes simplex viruses (HSV), measles viruses, rhabdoviruses, retroviruses, lentiviruses, Newcastle disease virus (NDV), poxviruses, and picornaviruses.

In various non-limiting examples, contemplated RNA molecules can be aptamers, such as Pegaptanib.

In some embodiments, contemplated DNA molecules can be DNA aptamers, DNAzymes, and oligonucleotides for anti-gene and antisense applications.

In some embodiments, contemplated small molecules are nonsense read-through drugs.

In various non-limiting examples, a gene, base, or RNA editor can include CRISPR, cytosine base editors (CBEs), adenine base editors (ABEs), TALEN base editors, zinc finger nucleases, antisense oligonucleotides, RNA trans-splicing molecules, and others.

In various non-limiting examples, contemplated cell therapies include stem cell transplantation.

In various aspects, this disclosure provides methods of testing for the efficacy of a therapeutic treatment using the retinal organoid model systems. The methods include administering to the retinal organoid model system a candidate therapeutic treatment for restoration of function of a gene encoding a structural component of the IPM; visualizing the photoreceptor outer segments within the IPM of the retinal organoid model system; and assessing the presence or absence of a change in the IPM, The presence of a change in production (i.e., an increase in production) and/or maintenance (i.e., prevention of loss) of microscopically visible photoreceptor outer segments within the IPM of the retinal organoid model system indicates that the therapeutic treatment was effective in restoration of function of the gene encoding a structural component of the IPM. The absence of a change in the production (i.e., no increase in production) and/or maintenance of microscopically visible photoreceptor visible outer segments indicates that the therapeutic treatment was not effective.

The therapeutic treatments can be administered to the retinal organoid model system using any suitable method. This includes, but is not limited to, nanoparticle drug delivery, membrane fusion, lipofection, ribonucleoprotein delivery, electroporation, local injection of the therapeutic treatment into the organoid, or addition of the therapeutic treatment to the media surrounding the organoid.

The methods of this disclosure can further comprise visualizing the IPM prior to the administration of the candidate therapeutic in order to assess the photoreceptor outer segments within the IPM prior to administration of the candidate therapeutic.

In various embodiments, visualizing of the IPM includes qualitatively observing or quantifying the presence, length, diameter and/or density of visible photoreceptor outer segments on the PR cell surface. In one embodiment, visualizing of the IPM includes identifying only the presence or absence of visible photoreceptor outer segments on the PR cell surface.

According to the methods of the present disclosure, assessing the state of the IPM can further include using qualitative observation or quantification of the visible outer segments on the PR cell surface to determine the presence or absence of a change in the IPM, wherein an increase in the presence or quantity of visible outer segments indicates the presence of a change in IPM of the retinal organoid model system. IPM status can also be assessed by looking at post translational modifications of proteins (i.e., proper glycosylation) of proteoglycans such as IMPG1 and 2.

According to further aspects of the present disclosure, the retinal organoid model systems can be used to model various genetic mutations and disorders and then test the efficacy of candidate therapeutic treatments. Efficacy of the candidate therapeutic treatments can be easily assessed using the clearly visible PR cell outer segment phenotype. In various embodiments of this aspect, the retinal organoid system can be engineered to comprise a predetermined genetic mutation that mimics one or more genetic mutations found in a genetic disease, as described herein elsewhere. The retinal organoid model system can then be used to test candidate therapeutic treatments. An effective treatment of the genetic mutation can be identified by a restoration of function of the gene encoding a structural component of the IPM.

### Examples

The Examples that follow are illustrative of specific embodiments of the disclosure, and various uses thereof. They are set forth for explanatory purposes only and should not be construed as limiting the scope of the disclosure in any way.

### Example 1: hPSC Retinal Organoid Establishment

Overview

In the present example, human pluripotent stem cells were used to establish retinal organoids.

Materials and Methods

The following hPSC cell lines were used: H9 IMPG2^{+/+}, H9 IMPG2^{-/-}, H9 IMPG2^{Y254C/Y254C}, iPSC IMPG2^{Y254C/A805(fs)Ter}, iPSC IMPG2^{Y254C/+}, iPSC IMPG2^{-/-}, and iPSC IMPG2

The hPSCs were maintained on Matrigel (WICell). To maintain the pluripotency of the hPSCs, mTeSR Plus (STEMCELL TECHNOLOGIES) was used. For passaging and making embryoid bodies (EBs), ReLeSR (STEMCELL TECHNOLOGIES) was used. EBs were then gradiantly transitioned from mTeSR plus to a neural induction medium (NIM; DMEM:F12 1:1, 1% N2 supplement, 1× MEM nonessential amino acids (MEM NEAA), 1× GlutaMAX (Thermo Fisher) and 2 mg/mL heparin (Sigma)) over the course of 4 days.

On day (d) 6, 1.5 nM BMP4 (R&D Systems) was added to fresh NIM, and on d7 EBs were plated on Matrigel® at a density of 200 EBs per well in a 6-well plate. Half the media was replaced with fresh NIM on d9, d12, and d15 to gradually dilute the BMP4, and on d16, the media was changed to Retinal Differentiation Medium (RDM; DMEM:F12 3:1, 2% B27 supplement, MEM NEAA, 1× antibiotic, anti-mycotic (Thermo Fisher) and 1× GlutaMAX). On d25-30, 3D optic vesicle-like structures (OVs) became apparent and were dissected with a MSP ophthalmic surgical knife (Surgical Specialties Corporation). Organoids were maintained in poly-HEMA-coated flasks (polyHEMA from Sigma) with twice-weekly feeding of 3D-RDM (DMEM:F12 3:1, 2% B27 supplement, 1× MEM NEAA, 1× antibiotic, anti-mycotic, and 1× GlutaMAX with 5% FBS, 100 μM taurine, 1:1000 chemically defined lipid supplement (11905031, Thermo Fisher)). Live cultures were imaged on a Nikon Ts2-FL equipped with a DS-Fi3 camera or on a Nikon Ts100 equipped with a QImaging CE CCD camera. All plasticware and reagents were from Thermo Fisher unless otherwise stated. All hPSC-ROs were differentiated to >200 days where IMPG2+/hPSC-RO displayed elongated photoreceptor outer segments (PR-OS; Capowski et al. 2019).

### Results

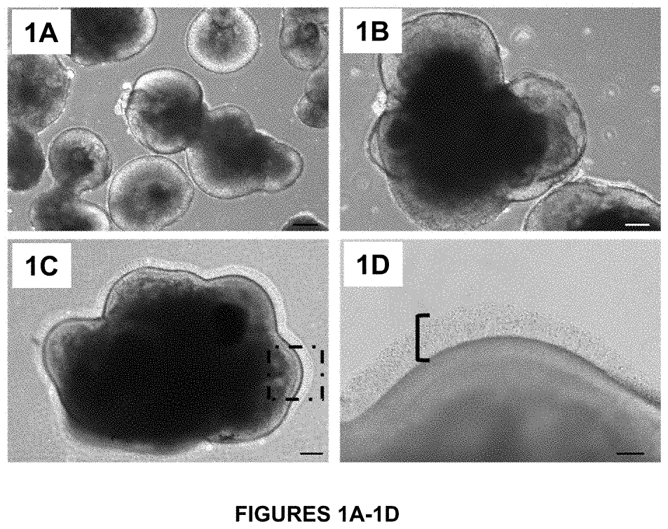

Light microscopic categorization of differentiating hPSC-ROs is shown in FIGS. 1A-1D. Stage 1 hPSC-ROs (FIG. 1A) are primarily composed of retinal progenitor cells and ganglion cells. Stage 2 hPSC-ROs (FIG. 1B) show an intermediate development with greater maturity of photoreceptor precursor cells. Stage 3 hPSC-ROs (FIGS. 1C and 1D) are characterized by the surface appearance of readily identifiable (via low magnification light microscopy) photoreceptor outer segments. FIG. 1D is a magnified image of the boxed area in FIG. 1C showing the photoreceptor outer segments (bracketed).

Conclusion

Photoreceptor outer segments in stage 3 hPSC-ROs are easily seen by low magnification light microscopy. Therefore, functional perturbations in genes necessary for producing photoreceptor outer segments should be easily identified.

### Example 2: IMPG2 Mutations and Photoreceptor (PR) Outer-Segment Maintenance

Overview

In the present example, single nucleotide polymorphism (SNP) modifications were used to generate mutations in IMPG2 to observe the effects on photoreceptor (PR) outer-segment (OS) maintenance.

Materials and Methods

To perform the single nucleotide polymorphism (SNP) modification, a protocol was used that utilized the single-strand oligonucleotide (ssODN) method discussed in Yang et al. (2013, Nucleic Acids Research, 41(19)). This approach was modified to fit within an existing CRISPR workflow published in Chen et al. (2015 Cell Stem Cell 17(2):233-44). In brief, single guide RNA (sgRNA) identification for the site of interest was performed using the CRISPOR design tool (www.crispor.tefor.net). The sgRNA sequences were then cloned into a pLentiCRISPR-V1 plasmid provided by the laboratory of Feng Zhang. To generate a Y254C mutation, a donor single-stranded oligo donor (ssODN) was used.

To generate H9 IMPG2^{-/-} and H9 IMPG2^{Y254C/Y254C} ROs, H9 IMPG2^{+/+} cells were used. To generate iPSC IMPG2^{Y254C/+} and iPSC IMPG2^{-/-} ROs, iPSC IMPG2^{Y254C/A805(fs)Ter} were used. Each cell line was grown according to the protocol in Example 1. To generate iPSC IMPG2^{+/+}, iPSC IMPG2^{Y254C/+} were used. Cells were cultured and electroporated as described in Chen et al. Following electroporation, cells were treated with puromycin to select for cells containing the pLentiCRISPRV1 plasmid. After puromycin selection, cells were cultured in MEF-conditioned hPSC media until colonies were visible for selection. Clonal populations were manually selected and isolated. Genomic DNA isolation was then performed to confirm successful integration of the ssODN and to perform off target analysis.

Results

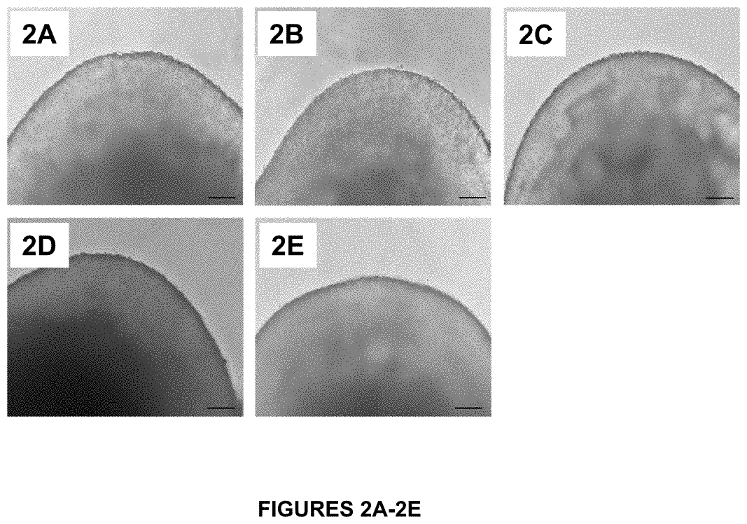

Differentiations of hPSC-ROs containing IMPG2 mutations result in a lack of photoreceptor outer segments as shown in FIGS. 2A-2E. hPSC lines were all differentiated >200 days, at which point photoreceptor outer segments are always present in aged-matched wildtype control hPSC lines (see FIG. 1). However, each line harboring IMPG2 mutations totally lacks outer segments as is easily determined by low magnification light microscopy (see FIG. 2A) iPSC IMPG2^{Y254C/A805(fs)Ter}, (FIG. 2B) iPSC IMPG2^{Y254C/+}, (FIG. 2C) iPSC IMPG2^{-/-}, (FIG. 2D) H9 IMPG2^{Y254C/Y254C}, and (FIG. 2E) H9 IMPG2^{-/-}.

Conclusion

Mutations in IMPG2 can result in an observable loss of function phenotype (a lack of photoreceptor outer segment maintenance) in retinal organoids. This shows an accelerated phenotype to what is seen in human patients as disease onset does not start until the second decade of life. However, surprisingly, after gene correction of a single allele, heterozygous IMPG2 hPSC-ROs still lacked photoreceptor outer segments (FIG. 2B), which contrasts with humans containing heterozygous IMPG2 mutations (MT) who have no clinical diagnosis of RP. The existence of a disease phenotype in heterozygous IMPG2 hPSC-ROs provides a powerful reference point/benchmark for expression levels needed to be achieved by therapeutic intervention to restore a wild-type (WT) phenotype. For example, restoration of a WT phenotype after treatment of a homozygous IMPG2 (MT/MT) model suggests that greater than 50% of normal protein expression has been achieved, which would be more than enough to restore a functional phenotype in a human RP patient since actual patients with heterozygous IPMG2 mutations have a normal phenotype. Indeed, restoration of a heterozygous state for other types of mutations associated with other diseases could also provide clinically meaningful results for patients with such diseases.

### Example 3: Restoration of PR Outer Segments in hPSC-ROs Containing IMPG2 Mutations

Overview

In the present example, single nucleotide polymorphism (SNP) modifications were used to determine whether a correction of induced mutations in IMPG2 could reverse the loss of function phenotype in retinal organoids by restoring visible photoreceptor outer segments.

Materials and Methods

Gene correction mutations were performed following the protocols outlined in Example 2 above using a different ssODN that reversed the mutation introduced into IMPG2 in Example 2 to return IMPG2 to wild type.

Results

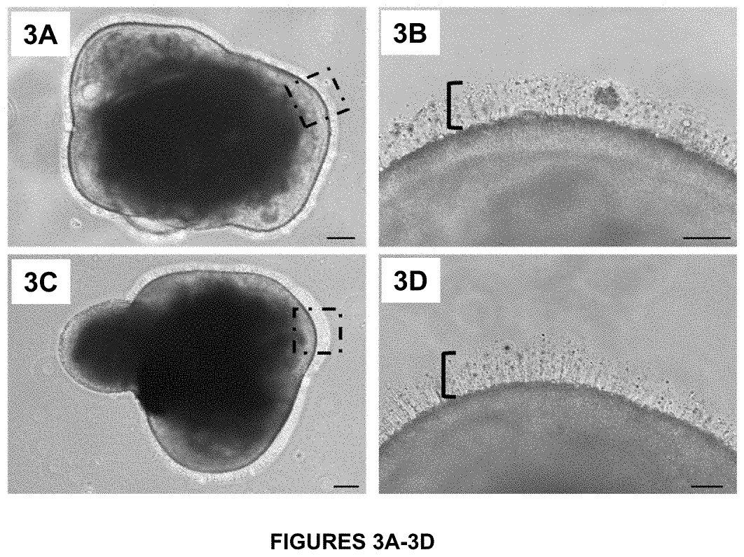

Stage 3 gene-corrected iPSC IMPG2^{+/+} ROs displayed photoreceptor outer segments (FIGS. 3A-3D). Gene-corrected IMPG2 mutant lines (i.e., conversion of mutant IMPG2 lines to wildtype IMPG2 status) were differentiated to >200 days. At this point these gene-corrected, isogenic iPSC IMPG2^{+/+} ROs demonstrated full restoration of photoreceptor outer segments along their surface (FIGS. 3A and 3B). A magnified image of the RO shown in panel FIG. 3A is shown in FIG. 3B. By way of comparison, the appearance of the restored outer segments in FIGS. 3A and 3B, is nearly indistinguishable from outer segments in unmodified, wildtype H9 ROs (FIGS. 3C and 3D).

Conclusion

Genetic manipulation to restore IMPG2 function leads to a restoration of visible outer segments in retinal organoids. These results suggest that restoration of IMPG2 function by gene therapy or by administration of other therapeutic agents to ROs harboring a genetically mutated IMPG2 gene could be used as a readily observable readout for determining the efficacy of such therapies. It is envisioned that any type of therapy that can restore IMPG2 function can be tested by this model system and that various types of genetic mutations, irrespective of gene source or associated disease, can be introduced into this model system for testing. Therefore, it is believed that the present model system can serve as a platform for testing candidate therapies and/or therapeutic agents for any genetic disease for which a genetic mutation associated with the disease can be identified and introduced into IMPG2 to result in a loss of IMPG2 function and loss of visible outer segments.

### Example 4: Evaluation of Novel Therapies for IMPG2-RP

Overview

In the present example, the level of correction needed to restore outer segments on the surface of ROs was explored by using mixed cultures of wild type and IMPG2 mutant hPSC lines. This example provided insight into the sensitivity of the model system for testing therapeutics.

Materials and Methods

IMPG2 mutant hPSCs lines are described in Example 2 above. hPSC photoreceptor reporter line (WA09 CRX^{+/TdTomato}) was generated according to Phillips et al. (2018, Stem Cells, 36(3): 313-324) and used as an IMPG2 wild type cell line. To create mixed wild type (WT) and IMPG2 mutant (MT) hPSC-ROs (i.e., hPSC-ROs made from mixed cultures of wild type and IMPG2 mutant hPSCs), wild type and IMPG2 mutant hPSCs lines were passaged as described above in Example 1. Once hPSCs were dissociated into single cells, cells were counted using a hemocytometer. The singularized hPSCs were then mixed in 15 mL conical tubes in three different wild type:IMPG2 mutant ratios: 1) 5:95 (to model a minimal level of gene augmentation); 2) 20:80 (to model a maximum level of gene augmentation possible in vivo with current viral delivery vectors); and 3) 50:50 (to model a targeted (expected) level of gene augmentation). The mixed cultures and specific ratios of cultures employed were designed to enable determination of the level of correction required (e.g., the degree of restoration of function needed) to restore outer segments on the surface of ROs.

Results

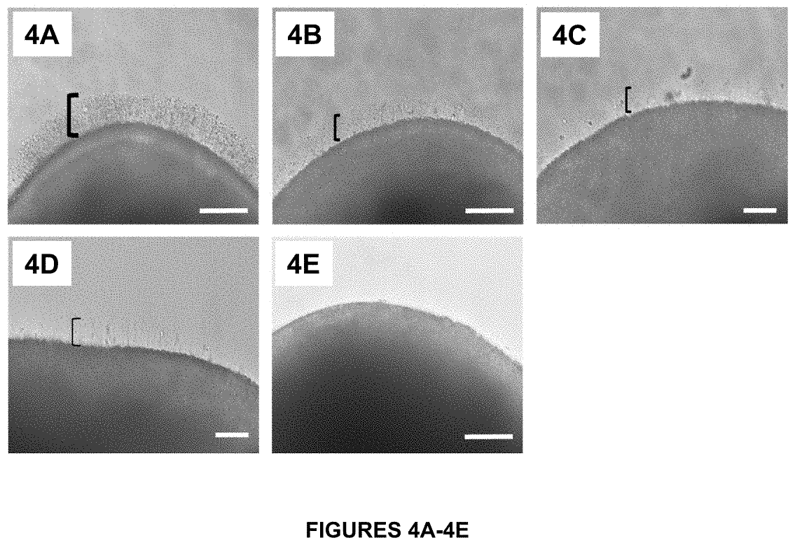

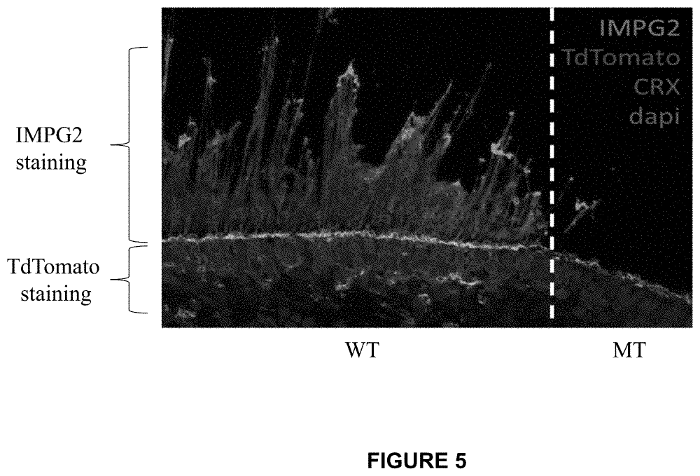

Light microscopic imaging of ROs derived from mixed cultures of wild type (WT) and mutant (MT) IMPG2 hPSCs are shown in FIGS. 4A-4E. FIG. 4A shows 100% WT control hPSC-RO with thick photoreceptor outer segments in brackets. FIG. 4B demonstrates a 50:50 WT:MT hPSC-RO culture displaying a moderate amount of photoreceptor outer segments, although reduced compared to 100% WT ROs. A further decrease in the presence of photoreceptor outer segments was observed with ratios of 20:80 (FIG. 4C) and 5:95 (FIG. 4D) WT:MT hPSC-RO cultures. FIG. 4E shows a 100% MT IMPG2 hPSC-RO control showing a complete absence of photoreceptor outer segments on the RO surface.

Fluorescent confocal imaging of a mixed WT (CRX-TdTomato):MT RO is shown in FIG. 5. The border between a WT region of a mixed RO (left of the white dashed line) and a MT region (right of the white dashed line) is shown. Note the presence and absence of IMPG2 expression in a variably thick surface layer of IPM in the WT or MT regions, respectively.

Conclusion

These results demonstrate that given a sufficient presence of wild type IMPG2, restoration of the IPM and photoreceptor outer segments is attainable.

### Example 5: H9 IMPG2^{-/-} Treatments with AAV5-IMPG2

Overview

In the present example, a gene augmentation approach was attempted to determine whether IMPG2 function could be restored in IMPG2 knockout H9 hPSC-ROs.

Materials and Methods

On day 150 of differentiation, IMPG2 knockout hPSC-ROs (as described above) were treated with 2^{c12} vp/mL of AAV5-IMPG2. The treated hPSC-ROs were monitored for the appearance of photoreceptor outer segments. After 50 days, the hPSC-ROs were cryosectioned and screened for IMPG2 expression via ICC. Immunostaining for a cone-specific protein, cone ARRESTIN-3 (AAR3) was used to visualize cone photoreceptors.

ICC analysis of AAV5-IMPG2 treated ROs was performed.

Results

Untreated, wild type H9 hPSC-ROs showed high levels of endogenous IMPG2 expression with photoreceptor outer segments marked by discontinuous (due to sectioning) ARR3 immunostaining. Knockout of IMPG2 in hPSC-ROs resulted in a complete lack of IMPG2 expression and photoreceptor outer segments in untreated hPSC-ROs. Treatment of IMPG2 knockout hPSC-RO with AAV5-IMPG2 showed some rescue of IMPG2 expression (data not shown).

Conclusion

These results demonstrate that restoration of function of IMPG2 via gene augmentation therapy can be monitored by the reappearance of photoreceptor outer segments in IMPG2 knockout hPSC-ROs. Therefore, it is concluded that additional approaches to restoring IMPG2 function can be tested using hPSC-RO lines that contain IMPG2 deleterious mutations. Specific candidate treatments can be tailored to specific types of mutations introduced into IMPG2.

### Example 6: Base Editing Approaches to Correct IMPG2 Function

Overview

In the present example, base editing approaches are considered for restoring IMPG2 function.

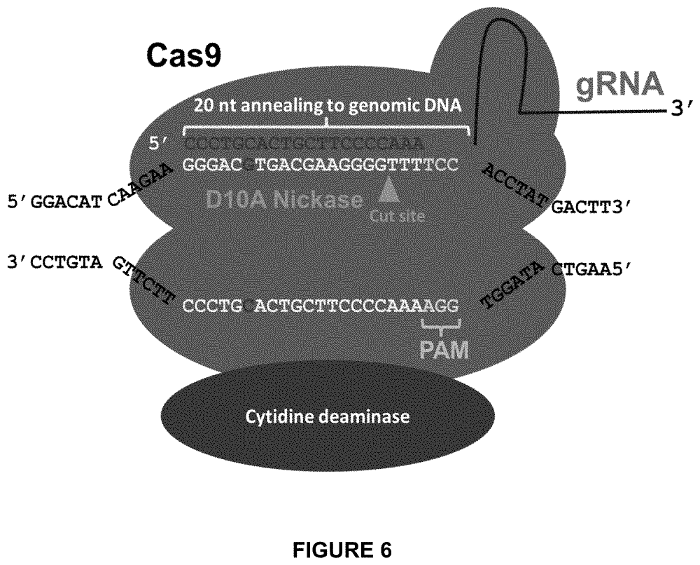

FIG. 6 shows possible base editing approach to correct for mutations in IMPG2. Shown in black is an example cytidine that causes the deleterious Y254C mutation in IMPG2. Using a Cas9-fused cytidine deaminase, this mutation can be targeted via the cytidine deaminase enzyme and can be converted from a guanine-cytosine pair to an adenine-thymine pair.

Significance

CRISPR/Cas9 genome editing has shown some ability to correct deleterious mutations via homology-directed repair (HDR). However, most double DNA stranded breaks that occur via CRISPR/Cas9 result in nonhomologous end joining (NHEJ), which does not result in mutation repair. CRISPR/Cas9 base editing also targets specific mutations for repair, but does not cause double stranded DNA breaks. Instead, the mutations are corrected though deaminase enzymes, allowing successful conversion of mutant base pairs.

All embodiments of any aspect of the disclosure can be used in combination, unless the context clearly dictates otherwise.

All patents and publications mentioned in this specification are herein incorporated by reference to the same extent as if each independent patent and publication was specifically and individually indicated to be incorporated by reference. Citation or identification of any reference in any section of this application shall not be construed as an admission that such reference is available as prior art to the present disclosure.

SEQUENCES

IMPG1 cDNA (SEQ ID NO: 1) NC_000006.12: c76072662-75921114 GENE ID: 3617
agacactgctacatgttcttcataaattaacaccctcataaaggtaaaccaagaaggttatcctcaatcatctggtatcaatatataattatt
tttcacatttctgttactttttaatgagatttgaggttgttctgtgattgttatcagaattaccaatgcacaaaagccagaatgtatttggaa
actagaagagctatttttgttttttggatttttctccaagttcaaggaaccaaagatatctccattaacatataccattctgaaactaaagac
atagacaatcccccaagaaatgaaacaactgaaagtactgaaaaaatgtacaaaatgtcaactatgagacgaatattcgatttggcaaagcat
cgaacaaaaagatccgcatttttcccaacgggggttaaagtctgtccacaggaatccatgaaacagattttagacagtcttcaagcttattat
agattgagagtgtgtcaggaagcagtatgggaagcatatcggatctttctggatcgcatccctgacacaggggaatatcaggactgggtcagc
atctgccagcaggagaccttctgcctctttgacattggaaaaaacttcagcaattcccaggagcacctggatcttctccagcagagaataaaa
cagagaagtttccctgacagaaaagatgaaatatctgcagagaagacattgggagagcctggtgaaaccattgtcatttcaacagatgttgcc
aacgtctcacttgggcctttccctctcactcctgatgacaccctcctcaatgaaattctcgataatacactcaacgacaccaagatgcctaca
acagaaagagaaacagaattcgctgtgttggaggagcagagggtggagctcagcgtctctctggtaaaccagaagttcaaggcagagctcgct
gactcccagtccccatattaccaggagctagcaggaaagtcccaacttcagatgcaaaagatatttaagaaacttccaggattcaaaaaaatc
catgtgttaggatttagaccaaagaaagaaaaagatggctcaagctccacagagatgcaacttacggccatctttaagagacacagtgcagaa
gcaaaaagccctgcaagtgacctcctgtcttttgattccaacaaaattgaaagtgaggaagtctatcatggaaccatggaggaggacaagcaa
ccagaaatctatctcacagctacagacctcaaaaggctgatcagcaaagcactagaggaagaacaatctttggatgtggggacaattcagttc
actgatgaaattgctggatcactgccagcctttggtcctgacacccaatcagagctgcccacatcttttgctgttataacagaggatgctact
ttgagtccagaacttcctcctgttgaaccccagcttgagacagtggacggagcagagcatggtctacctgacacttcttggtctccacctgct
atggcctctacctccctgtcagaagctccacctttctttatggcatcaagcatcttctctctgactgatcaaggcaccacagatacaatggcc
actgaccagacaatgctagtaccagggctcaccatccccaccagtgattattctgcaatcagccaactggctctgggaatttcacatccacct
gcatcttcagatgacagccgatcaagtgcaggtggcgaagatatggtcagacacctagatgaaatggatctgtctgacactcctgccccatct
gaggtaccagagctcagcgaatatgtttctgtcccagatcatttcttggaggataccactcctgtctcagctttacagtatatcaccactagt
tctatgaccattgcccccaagggccgagagctggtagtgttcttcagtctgcgtgttgctaacatggccttctccaacgacctgttcaacaag
agctctctggagtaccgagctctggagcaacaattcacacagctgctggttccatatctacgatccaatcttacaggatttaagcaacttgaa
atacttaacttcagaaacgggagtgtgattgtgaatagcaaaatgaagtttgctaagtcagtgccgtataacctcaccaaggctgtgcacggg
gtcttggaggattttcgttctgctgcagcccaacaactccatctggaaatagacagctactctctcaacattgaaccagctgatcaagcagat
ccctgcaagttcctggcctgcggcgaatttgcccaatgtgtaaagaacgaacggactgaggaagcggagtgtcgctgcaaaccaggatatgac
agccaggggagcctggacggtctggaaccaggcctctgtggccctggcacaaaggaatgcgaggtcctccagggaaagggagctccatgcagg
ttgccagatcactctgaaaatcaagcatacaaaactagtgttaaaaagttccaaaatcaacaaaataacaaggtaatcagtaaaagaaattct
gaattactgaccgtagaatatgaagaatttaaccatcaagattgggaaggaaattaaaaactgaaaatgtacaattatcatttaggctatctc
aagagagatgatttgccttctcaaggaaaatggagacaggcatattcatgggtcatcaaaatccagacatacagtcaacactgagaatcagca
cacaccatatttcaaatatagaagagtcatgtacttggcaaccagtaaattctgaagaaaaagacacttacttattattaaaaccccaaatgc
aatcagcgaaacatatttttactattcttggatgatagtcaaaatgatcataagccaggtttgcttccaccttccctgaaaattttactcaca
gatcatttgcaacaagcatagcttacttattgtttagggactgaacaatttattgggaagcaaactctttatatgctagaaagtacatttaaa
agatgactacttacgcagggagatgcaggtctctctaaacgcatgaatgtatgtagtgtgtaggcactgtagtgagtgtatatatgctccaca
ctacgtctgataaacacaaacctcagtattcagttattaggcacactagttttatacgcaactactgcttacatagtagactgttttgttgcc
aataatctttgaattgttctttaaaagaaactgaggttcagatacacataccatggaaaaatcttacttttcttgttactacacaaagctatt
ttaaagaagatgctatgttgggagaagggcgaagttgtactatatgacataatcaattcctgtcttccaccacagatgaacaatgtcttccta
taataacttcagaatatttcctcacaccaactttagtgtgtgtatacctagactggcatcaatgtaatccaattcagtccattttttatgtgc
tgcttaatgaaaatgaatctgtctctatctctttcaaatgttctaatatcagatatatttgctacaaaccttaatttcaataaagtaaaagtg
atatactca
IMPG2 cDNA (SEQ ID NO: 2) NC_000003,12: c101320575-101222546 GENE ID: 50939
atgattatgtttcctctttttgggaagatttctctgggtattttgatatttgtcctgatagaaggagactttccatcattaacagcacaaacc
tacttatctatagaggagatccaagaacccaagagtgcagtttcttttctcctgcctgaagaatcaacagacctttctctagctaccaaaaag
aaacagcctctggaccgcagagaaactgaaagacagtggttaatcagaaggcggagatctattctgtttcctaatggagtgaaaatctgccca
gatgaaagtgttgcagaggctgtggcaaatcatgtgaagtattttaaagtccgagtgtgtcaggaagctgtctgggaagccttcaggactttt
tgggatcgacttcctgggcgtgaggaatatcattactggatgaatttgtgtgaggatggagtcacaagtatatttgaaatgggcacaaatttt
agtgaatctgtggaacatagaagcttaatcatgaagaaactgacttatgcaaaggaaactgtaagcagctctgaactgtcttctccagttcct
gttggtgatacttcaacattgggagacactactctcagtgttccacatccagaggtggacgcctatgaaggtgcctcagagagcagcttggaa
aggccagaggagagtattagcaatgaaattgagaatgtgatagaagaagccacaaaaccagcaggtgaacagattgcagaattcagtatccac
cttttggggaagcagtacagggaagaactacaggattcctccagctttcaccaccagcaccttgaagaagaatttatttcagaggttgaaaat
gcatttactgggttaccaggctacaaggaaattcgtgtacttgaatttaggtcccccaaggaaaatgacagtggcgtagatgtttactatgca
gttaccttcaatggtgaggccatcagcaataccacctgggacctcattagccttcactccaacaaggtggaaaaccatggccttgtggaactg
gatgataaacccactgttgtttatacaatcagtaacttcagagattatattgctgagacattgcagcagaattttttgctggggaactcttcc
ttgaatccagatcctgattccctgcagcttatcaatgtgagaggagttttgcgtcaccaaactgaagatctagtttggaacacccaaagttca
agtcttcaggcaacgccgtcatctattctggataatacctttcaagctgcatggccctcagcagatgaatccatcaccagcagtattccacca
cttgatttcagctctggtcctccctcagccactggcagggaactctggtcagaaagtcctttgggtgatttagtgtctacacacaaattagcc
tttccctcgaagatgggcctcagctcttccccagaggttttagaggttagcagcttgactcttcattctgtcaccccggcagtgcttcagact
ggcttgcctgtggcttctgaggaaaggacttctggatctcacttggtagaagatggattagccaatgttgaagagtcagaagattttctttct
attgattcattgccttcaagttcattcactcaacctgtgccaaaagaaacaataccatccatggaagactctgatgtgtccttaacatcttca
ccatatctgacctcttctataccttttggcttggactccttgacctccaaagtcaaagaccaattaaaagtgagccctttcctgccagatgca
tccatggaaaaagagttaatatttgacggtggtttaggttcagggtctgggcaaaaggtagatctgattacttggccatggagtgagacttca
tcagagaagagcgctgaaccactgtccaagccgtggcttgaagatgatgattcacttttgccagctgagattgaagacaagaaactagtttta
gttgacaaaatggattccacagaccaaattagtaagcactcaaaatatgaacatgatgacagatccacacactttccagaggaagagcctctt
agtgggcctgctgtgcccatcttcgcagatactgcagctgaatctgcgtctctaaccctccccaagcacatatcagaagtacctggtgttgat
gattactcagttaccaaagcacctcttatactgacatctgtagcaatctctgcctctactgataaatcagatcaggcagatgccatcctaagg
gaggatatggaacaaattactgagtcatccaactatgaatggtttgacagtgaggtttcaatggtaaagccagatatgcaaactttgtggact
atattgccagaatcagagagagtttggacaagaacttcttccctagagaaattgtccagagacatattggcaagtacaccacagagtgctgac
aggctctggttatctgtgacacagtctaccaaattgcctccaaccacaatctccaccctgctagaggatgaagtaattatgggtgtacaggat
atttcgttagaactggaccggataggcacagattactatcagcctgagcaagtccaagagcaaaatggcaaggttggtagttatgtggaaatg
tcaacaagtgttcactccacagagatggttagtgtggcttggcccacagaaggaggagatgacttgagttatacccagacttcaggagctttg
gtggttttcttcagcctccgagtgactaacatgatgttttcagaagatctgtttaataaaaactccttggagtataaagccctggagcaaaga
ttcttagaattgctggttccctatctccagtcaaatctcacggggttccagaacttagaaatcctcaacttcagaaatggcagcattgtggtg
aacagtcgaatgaagtttgccaattctgtccctcctaacgtcaacaatgcggtgtacatgattctggaagacttttgtaccactgcctacaat
accatgaacttggctattgataaatactctcttgatgtggaatcaggtgatgaagccaacccttgcaagtttcaggcctgtaatgaattttca
gagtgtctggtcaacccctggagtggagaagcaaagtgcagatgcttccctggatacctgagtgtggaagaacggccctgtcagagtctctgt
gacctacagcctgacttctgcttgaatgatggaaagtgtgacattatgcctgggcacggggccatttgtaggtgccgggtgggtgagaactgg
tggtaccgaggcaagcactgtgaggaatttgtgtctgagcccgtgatcataggcatcactattgcctccgtggttggacttcttgtcatcttt
tctgctatcatctacttcttcatcaggactctccaagcacaccatgacaggagtgaaagagagagtcccttcagtggctccagcaggcagcct
gacagcctctcatctattgagaatgctgtgaagtacaaccccgtgtatgaaagtcacagggctggatgtgagaagtatgagggaccctatcct
cagcatcccttctacagctctgctagcggagacgtgattggtgggctgagcagagaagaaatcagacagatgtatgagagcagtgagctttcc
agagaggaaattcaagagagaatgagagttttggaactgtatgccaatgatcctgagtttgcagcttttgtgagagagcaacaagtggaagag
gtttaa
Example annealing sequence for Cas9-fused cytidine deaminase gene editing approach
(SEQ ID NO: 3)-ccctgcactgcttccccaaa
Example 5′-3′ IMPG2 gene segment targeted by Cas9-fused cytidine deaminase gene editing
approach (SEQ ID NO: 4)-ggacatcaagaagggacgtgacgaaggggttttcc
Example 3′-5′ IMPG2 gene segment targeted by Cas9-fused cytidine deaminase gene editing
approach (SEQ ID NO: 5)-cctgtagttcttccctgcactgcttccccaaaagg

We claim:

1. A retinal organoid model system, comprising:
a population of human pluripotent stem cell (hPSC)—derived photoreceptor (PR) cells,
wherein the PR cells are adapted to elaborate an interphotoreceptor matrix (IPM) with visible outer segments on a surface thereof upon restoration of function of a gene encoding a structural component of the IPM.

2. The retinal organoid model system of claim 1, wherein the hPSC is a human embryonic stem cell (hESC) or a human induced pluripotent stem cell (hiPSC).

3. The retinal organoid model system of claim 1, wherein the function of the gene is restorable by administration of a therapeutic treatment to the retinal organoid.

4. The retinal organoid model system of claim 3, wherein the therapeutic treatment comprises a protein, a virus, a RNA molecule, a DNA molecule, or a small molecule.

5. The retinal organoid model system of claim 4, wherein the therapeutic treatment further comprises a gene editor, a base editor, an RNA editor, a small molecule targeting DNA/RNA, or a cell therapy.

6. The retinal organoid model system of claim 2, wherein the hPSC is a hiPSC that is either a) a patient-derived hiPSC comprising a naturally occurring mutation in a gene encoding a structural component of the IPM or b) a recombinant hiPSC comprising a genetically engineered mutation in at least one allele in the gene encoding a structural component of the IPM.

7. The retinal organoid model system of claim 6, wherein the naturally occurring mutation is one or more of a missense mutation, a nonsense mutation, a frameshift mutation, a cryptic slice variant mutation, a coding mutation, or non-coding mutation.

8. The retinal organoid model system of claim 2, wherein the hPSC is a hESC selected from the group consisting of hESC is H9, H1, H7, BGO1, BG02, HES-3, HES-2, HSF-6, HUES9, HUES7, and 16.

9. The retinal organoid model system of claim 8, wherein the hESC is a recombinant hESC comprising a genetically engineered mutation in at least one allele in the gene encoding a structural component of the IPM.

10. The retinal organoid model system of claim 9, wherein the genetically engineered mutation is one or more of a missense mutation, a nonsense mutation, a frameshift mutation, a cryptic slice variant mutation, a coding mutation, or non-coding mutation.

11. The retinal organoid model system of claim 1, wherein the gene encoding a structural component of the IPM is IMPG1 or IMPG2.

12. The retinal organoid model system of claim 11, wherein the gene encoding a structural component of the IPM is IMPG2.

13. A retinal organoid model system, comprising:
a population of human pluripotent stem cell (hPSC)—derived photoreceptor (PR) cells,
wherein the PR cells comprise a recombinant gene encoding a structural component of an interphotoreceptor matrix (IPM),
wherein the gene comprises at least one of
i) a first non-functional allele comprising a first engineered genetic mutation, and/or
ii) a second non-functional allele comprising a second engineered genetic mutation, and
wherein restoration of function of at least one of the first and second alleles produces an IPM containing visible outer segments on a surface of the PR cells.

14. The retinal organoid model system of claim 13, wherein the function of the gene is restored by administration of a therapeutic treatment to the retinal organoid, wherein the therapeutic treatment comprises a protein, a virus, a RNA molecule, a DNA molecule, or a small molecule.

15. A method of testing for an effective therapeutic treatment for a genetic mutation, comprising:
a. producing a retinal organoid model system comprising a population of human pluripotent stem cell (hPSC)—derived photoreceptor (PR) cells adapted to express an interphotoreceptor matrix (IPM) with visible outer segments on a surface thereof upon restoration of function of a gene encoding a structural component of the IPM,
wherein the gene encoding the structural component of the IPM comprises a predetermined genetic mutation;
b. administering a candidate therapeutic treatment to the retinal organoid model system; and
c. assessing the retinal organoid model system for presence or absence of visible outer segments within the IPM,
wherein the presence of visible outer segments of the IPM indicates that the candidate therapeutic treatment restores the function of the gene encoding the structural component of the IPM by effectively treating the predetermined genetic mutation.

16. The method of claim 15, wherein the candidate therapeutic treatment comprises a protein, a virus, a RNA molecule, a DNA molecule, a gene therapy, a small molecule, a gene editor, a base editor, an RNA editor, a small molecule targeting DNA/RNA, a cell therapy, a genome or base editing technology, a nanoparticle, or a cellular delivery mechanism.

17. The method of of claim 15, wherein the candidate therapeutic treatment is a candidate for the treatment of a genetic disease.

18. The method of claim 17, wherein the genetic disease is cystic fibrosis, sickle-cell anemia, hemochromatosis, Huntington's disease, Duchenne's muscular dystrophy Tay-Sachs disease, Angelman syndrome, Ankylosing spondylitis, Marfan syndrome, or Thalassemia.

19. A method of making a retinal organoid model system, comprising:
a. engineering one or more genetic mutations in a gene encoding a structural component of the interphotoreceptor matrix (IPM) in a population of human pluripotent stem cells (hPSCs); and
b. inducing the hPSCs to differentiate along a retinal lineage, wherein the differentiation results in a three-dimensional (3D) retinal organoid comprising photoreceptor (PR) cells adapted to express visible outer segments within the IPM on a surface thereof upon restoration of function of the gene encoding a structural component of the IPM.

20. A method of making a retinal organoid model system, comprising:
a. obtaining a tissue sample from a subject having a mutation in a gene encoding a structural component of the interphotoreceptor matrix (IPM);
b. establishing a population of human induced pluripotent stem cells (hiPSCs) from the tissue sample; and
c. inducing the hiPSCs to differentiate along a retinal lineage, wherein the differentiation results in a three-dimensional (3D) retinal organoid comprising photoreceptor (PR) cells,
wherein the PR cells are adapted to express an interphotoreceptor matrix (IPM) structural protein, and
wherein restoration of the IPM structure allows the production and maintenance of visible photoreceptor outer segments on the surface of the retinal organoids.
